(19) United States
(12) Patent Application Publication
(10) Pub. No.: US 2021/0030410 A1
(43) Pub. Date: Feb. 4, 2021

(54) ANCHOR FOR SURGICAL TISSUE REPAIRS

(71) Applicant: H & B ELECTRONIC GMBH & CO KG, Deckenpfronn (DE)

(72) Inventors: Tobias MORLOK, Mötzingen (DE); Wilfried WEBER, Schopfloch (DE); Dariusz PETRY, Aidlingen (DE); Wolfgang STAUSS, Rangendingen (DE)

(73) Assignee: H & B Electronic GmbH & Co KG, Deckenpfronn (DE)

(21) Appl. No.: 16/645,061

(22) PCT Filed: Aug. 15, 2018
(86) PCT No.: PCT/EP2018/072146

§ 371 Date: Mar. 6, 2020

(30) Foreign Application Priority Data
Sep. 27, 2017 (DE) 20 2017 105 870.0

(51) Int. Cl. A61B 17/04

(52) U.S. Cl. CPC A61B 17/0401; A61B 2017/00862

(57) ABSTRACT

An anchor for surgical repair of a tissue, which anchor, for its placement on the tissue to be repaired, is movable along a hollow needle and connectable to a suture element, and it has at least two anchor portions displaceable relative to each other and connected to each other by an axle arrangement, via which the anchor portions are pivotable relative to each other between a retracted position and a deployed position. Provision is made that the axle arrangement has an axle element provided on the first anchor portion and a guide recess provided on the second anchor portion, by which the first anchor portion is held rotatably and linearly displaceably relative to the second anchor portion, and the anchor portions are displaceable from the retracted position to the deployed position by a rotation movement and are displaceable from said deployed position to a rotationally blocked end position by a linear movement.

The invention relates to an anchor for surgical tissue repair according to the preamble of claim 1, such as in particular for repairing a meniscus tear, a ligament or tendon. In this regard, for its placement on a tissue to be repaired, the anchor can be moved along a hollow needle and in addition is or can be connected with a suture element. Furthermore, the anchor has at least two anchor portions displaceable relative to each other which are connected to each other by an axle arrangement. Via this axle arrangement, the anchor portions can be pivoted relative to each other between a retracted position and a deployed position.

From DE 20 2015 002244 U1 is known an anchor arrangement for surgical tissue repair. The anchor arrangement has in this regard at least one first anchor and one second anchor which for the respective placement on the tissue to be repaired can be moved along a hollow needle and ejected therefrom. The at least two anchors are in this regard connected with each other via a suture element. For repairing the tissue concerned, in this regard at least one part, connecting the two anchors, of the suture element can be shortened, as a result of which for example a tear in the tissue can be closed. In this regard, two anchor portions are provided at the anchors which are connected via a joint axle. Thus, the anchors can be displaced at least partially transversely to the hollow needle or to a through opening, generated by means of a hollow needle, in the tissue. By means of the displacement of the anchor portions, it can thus be prevented that the anchors are drawn back again through the through-opening when tension is applied to the suture element.

In the case of the known anchors, the anchor portions are thereby held securely in the deployed position in that they abut on the tissue concerned under tension. It is the task of the invention to further stabilise the deployed position of the two anchor portions in the case of a generic anchor.

This task is solved by an anchor with the characteristics of claim 1. In this regard, the axle arrangement has an axle element provided on the first anchor portion and a guide recess provided on the second anchor portion, by means of which the first anchor portion is held rotatably and linearly displaceably on the second anchor portion. In this regard, the anchor portions are displaceable out of the retracted position by means of a rotational movement into the deployed position, and from this by means of a linear movement into a rotationally blocked end position. The connection between the two anchor portions thus has two degrees of freedom, which facilitates on the one hand a rotation movement for opening the two anchor portions into the deployed position and additionally a longitudinal movement for the mutual displacement into the end position, in which a rotation movement back into the retracted position is blocked. Thus, the anchor, after the setting, independently of its positioning on the tissue, can be securely held in the opened end position, in order to rule out an accidental re-entry into a puncture opening of the tissue and by means of the suture element to be able to apply a relatively high tensile force onto the tissue portion to be repaired.

In an especially advantageous embodiment, the axle element is designed integrally with the first anchor portion. Hereby, the rotatable connection, displaceable in longitudinal direction, between the two anchor portions can also be achieved in a relatively stable manner even in the case of small total dimensions.

Alternatively, it is advantageous when the axle element is formed by a pin which can be connected with the first anchor portion, which pin is in particular manufactured separately to the first anchor portion and which can be inserted into an axle reception introduced into the first anchor portion. Thereby, a relatively inexpensive manufacture of the axle element and the anchor as a whole is possible.

Here, it is advantageous when the guide recess has an elastically extendable introduction opening via which the axle element can be introduced and fixed on the first anchor portion in the manner of a latch connection. The elastically extendable introduction opening thus facilitates a simple mounting of the axle element on the first axle portion.

Advantageously, the guide recess is formed by two longitudinal openings disposed parallel, which are introduced into two portions, spaced from one another, of the second anchor portion. These anchor portions are in this case spaced from one another so far that the axle element can be inserted between them and by turning about 90° can be brought into engagement at both ends with the longitudinal openings, wherein the axle element is guidedly displaceable along the longitudinal openings. This embodiment facilitates an especially simple rotatable and linearly displaceable connection of the two anchor portions.

In addition, it is advantageous when the axle element and the guide recess are provided on or adjacent to an end of the respective anchor portion, such that the anchor after the unfolding, or in the end position, has a maximised longitudinal extension.

Advantageously, the guide recess has an elongated hole along which the axle element can be displaced into the end position, as a result which after reaching the deployed position a secure mutual guidance of the anchor portions into the end position can be guaranteed.

Furthermore, it is advantageous when the axle element in the retracted position can be latched at the elongated hole, in order during the unfolding movement to be able to guarantee a pure rotational movement or pivotal movement between the anchor portions.

In addition, it is advantageous when the axle element in the end position can be latched at the elongated hole in the in order to be able to rule out an accidental displacement out of the end position or an undesirable folding-together of the two anchor portions.

In a further advantageous embodiment, the guide recess has a form-fit means, which in the end position interacts with a form-fit counter means of the axle element, such that after the reaching of the end position an accidental rotational movement can be ruled out.

Furthermore, it is advantageous when the first anchor portion has at least one first abutment surface which in the end position comes into abutment with a second abutment surface of the second anchor portion. In particular in the presence of a certain preload directed against each other, the two anchor portions can thus in the end position be positioned in a predetermined position in a stable manner against one another.

Advantageously, both anchor portions have respectively one receiving opening at which the suture element can respectively be redirected or fixed. By placing the anchor against an abutment or against the tissue to be handled, and simultaneous application of tensile forces by means of the receiving openings, hereby moments and longitudinal displacement forces can be generated in a predetermined working direction, by means of which the anchor after the setting can be securely unfolded and moved into the end position. In addition, the anchor portions can be held by means of the respective receiving opening on the suture element in an unlosable manner. In this manner, it can be ensured that the anchor portions remain on the suture element in particular also in the case of damage to the axle element or to the guide recess.

In addition it is advantageous when the anchor portions in the retracted position have a composite cross-section which extends completely within a virtual enveloping circle with a diameter of less than 2 mm, in order to facilitate a problem-free piercing and penetrating of the tissue to be repaired by means of a needle.

Moreover, it is advantageous when the anchor portions end position have a total length of less than 10 mm, in order to guarantee that the anchor remains in the body of the patient concerned in a manner which is as disturbance-free as possible and which causes as little damage to tissue as possible.

Further, the above-mentioned task is solved by an anchor setting arrangement having an anchor accommodated in a needle in one of the above-mentioned embodiments and a suture element, wherein a tensile force can be applied to the at least two anchor portions by means of the suture element, in order first to apply an unfolding moment to the anchor portions during or after the setting, which moves it into the deployed position, and subsequently to apply longitudinal forces, which move the anchor portions by means of a linear movement into the end position.

In this regard it is advantageous when the tensile force can be generated by means of a tension sleeve connected with the suture element, which is displaceably held on the needle or on a setting device connected with the needle, and to which a preload can be applied. Thus, the anchor portions can already prior to and during the setting procedure be preloaded into the deployed position, such that when they emerge out of the needle they can be directly unfolded and fixed on one another in the end position. Thus, an accidental re-entry of the anchor into an entry opening in the tissue created during the setting procedure can be substantially ruled out.

Advantageously, in addition to the tensile force which can be applied by means of the suture element to both anchor portions, a pressing force directed in the opposite direction offset to the tensile force can be applied to said anchor portions in order to generate an unfolding moment, by means of which the anchor portions can be brought in a targeted manner into the deployed position.

In this regard, it is advantageous when the pressing force is generated by means of a displaceable ejector in the needle, as a result of which the ejection movement of the ejector can be used simultaneously to generate the unfolding moment.

Advantageously, the suture element engages on the two anchor portions with tensile forces directed obliquely towards one another. By means of this oblique orientation of for example two portions, to extend towards each other obliquely with respect to the anchor portions, of the suture element, it is possible to generate in addition to the unfolding moment also force components directed towards one another, by means of which the anchor portions can be moved out of the deployed position in longitudinal direction into the mutual end position.

In a further advantageous embodiment, a lifting suture element is provided for moving the anchor portions out of the retracted position into the deployed position and/or the end position as well as a holding suture element for fastening the anchor with respect to the tissue to be repaired or for connecting with a further anchor. By means of this use of two separated suture elements for different functions, said suture elements can be looped through the anchors and adapted to the respective function. Thus, it is for example possible to form the lifting suture element, which does not need to assume any holding function, by means of a relatively thin strand, which in particular can be rapidly reabsorbed.

It is noted that all above described characteristics of the subject matter according to the invention can be exchanged or combined among each other, inasmuch as an exchange or a combination thereof is not excluded for technical reasons.

In the figures, an exemplary embodiment of the invention is shown.

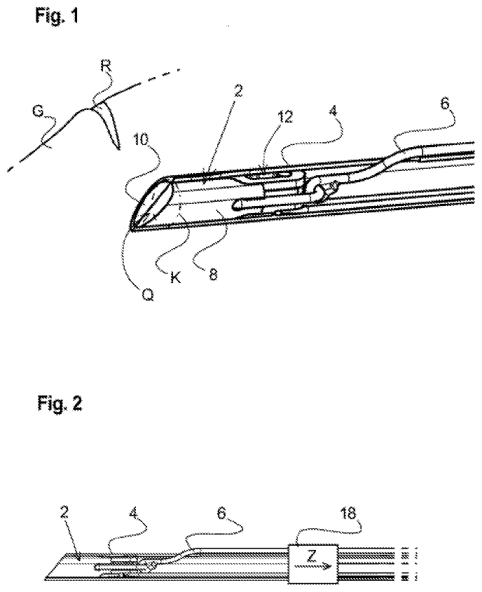

FIG. 1 shows a perspective view of an anchor according to the invention, accommodated in a partially cut-free hollow needle, in a retracted position,

FIG. 2 shows a side view of the anchor according to FIG. 1,

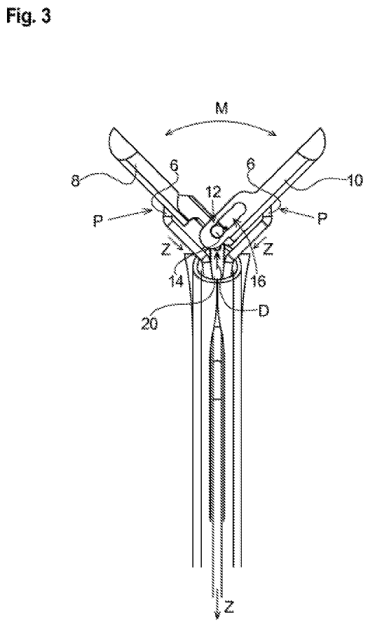

FIG. 3 shows a view of the anchor according to FIG. 1 emerging out of the hollow needle,

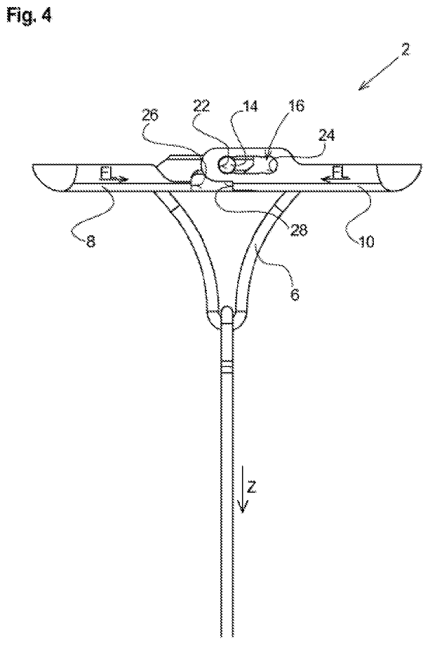

FIG. 4 shows a view of the anchor according to FIG. 1 in a deployed position,

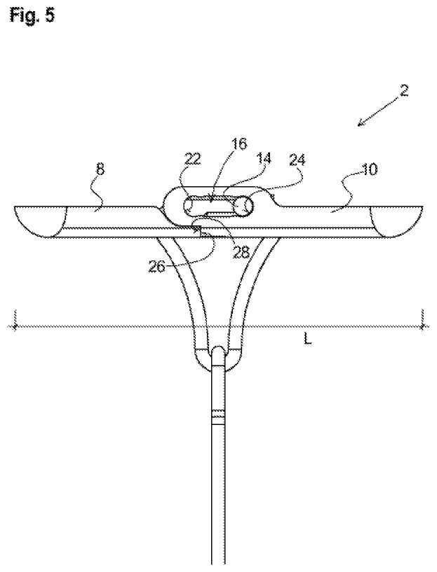

FIG. 5 shows a view of the anchor according to FIG. 1 in an end position,

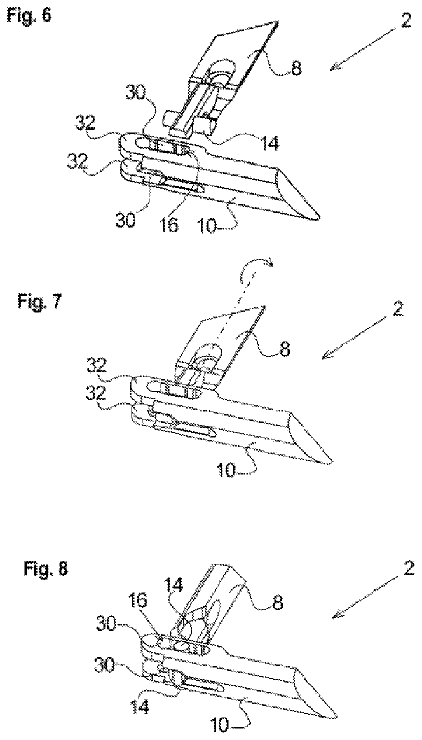

FIG. 6 shows a perspective view of the anchor according to FIG. 1 with separated anchor portions,

FIG. 7 shows a perspective view of the anchor according to FIG. 6 while an axle element is inserted between two longitudinal openings,

FIG. 8 shows a perspective view of the anchor according to FIG. 7 while the axle element engages into the longitudinal openings,

FIG. 9 shows an exploded perspective view of an alternative embodiment of the anchor with a pin-shaped axle element,

FIG. 10 shows a perspective view of the anchor according to FIG. 9 with joined-together anchor portions,

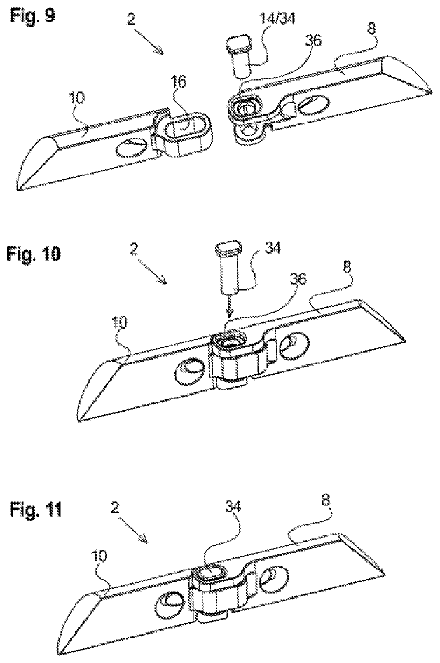

FIG. 11 shows a perspective view of the anchor according to FIG. 9 with mounted axle arrangement,

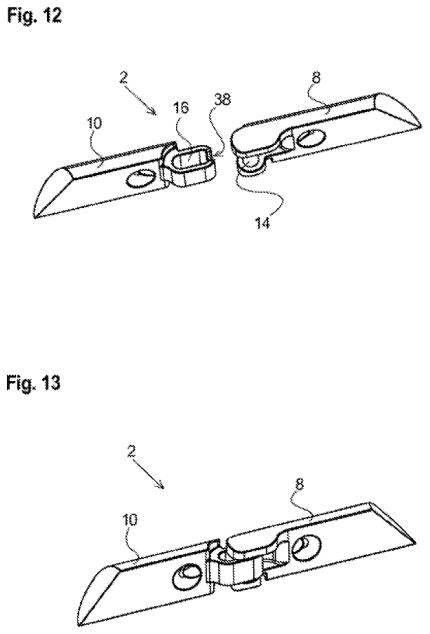

FIG. 12 shows a perspective view of a further alternative embodiment of the anchor with a lateral axis arrangement in a separated state,

FIG. 13 shows a perspective view of the anchor according to FIG. 12 in a joined-together state,

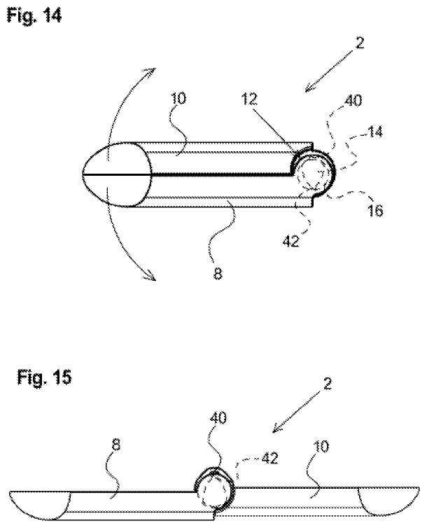

FIG. 14 shows a view of a further alternative embodiment of the anchor with a guide recess, having form-fit means, in the retracted position,

FIG. 15 shows a view of the anchor according to FIG. 14 in the deployed position,

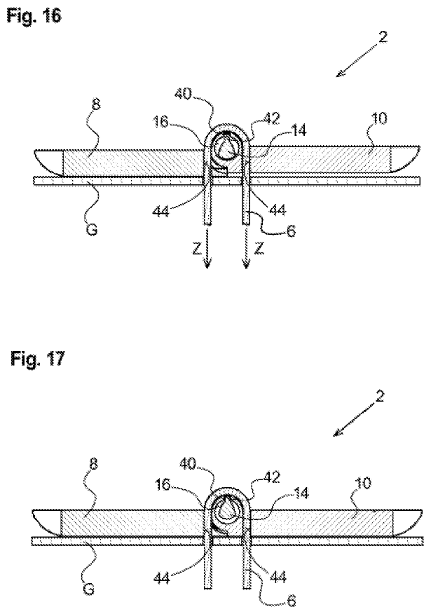

FIG. 16 shows a cut view of the anchor according to FIG. 15 while it is laid onto a tissue to be repaired,

FIG. 17 shows a cut view of the anchor according to FIG. 16 in the end position,

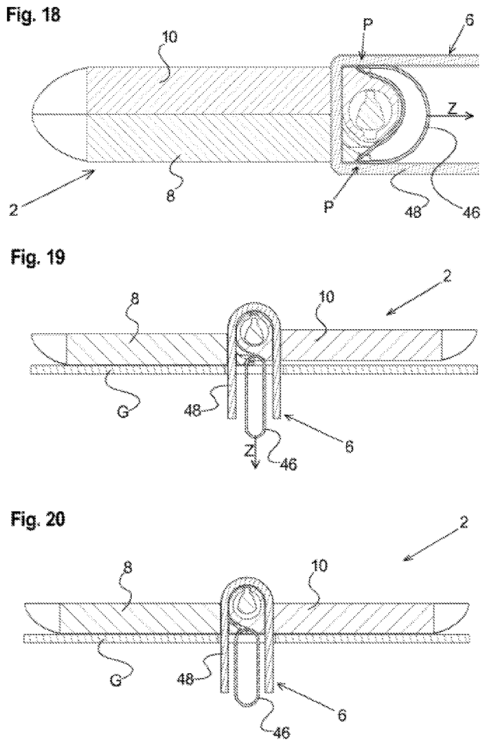

FIG. 18 shows a cut view of the anchor according to FIG. 15 in the retracted position with an alternative embodiment of the suture element,

FIG. 19 shows a cut view of the anchor according to FIG. 18 in the deployed position and

FIG. 20 shows a cut view of the anchor according to FIG. 18 in the end position.

FIG. 1 shows an anchor 2 for the surgical repair of tissue of a meniscus tear, a ligament or a tendon. To this end, the anchor 2 is designed such that it can be moved along a hollow needle 4 and positioned out of it on the tissue G to be repaired. Additionally, the anchor 2 is connected with a suture element 6, in particular in the form of a suture, by means of which the anchor 2 can be connected with a further anchor or by means of which a tensile force can be applied to the anchor. For repairing the concerned tissue G, for example a portion, disposed between two set anchors 2, of the suture element 6 can be shortened by drawing it together, as a result of which for example a tear R in the tissue G can be closed, in order to facilitate the tissue G growing together at this site.

As seen from FIG. 1, the anchor 2 has a first anchor portion 8 and a second anchor portion 10, which are connected with each other by means of an axle arrangement 12. In the shown retracted position, the two anchor portions 8, 10 lie against one another aligned parallel to one another, such that they form a composite cross-section Q which extends completely within a enveloping circle K which has a diameter of less than 2 mm. Thus, the anchor 2 can in the retracted position be accommodated within the relatively thin hollow needle 4 and be moved along it. Preferably, in this regard, both the composite cross-section Q of the anchor 2 and the hollow needle 4 have a profile which deviates from a circular shape, in order to be able to receive and set the anchor 2 in a predetermined rotational position.

As can be seen from FIG. 2, a tension sleeve 18 is provided on the hollow needle 4, by means of which a tensile force Z can be applied to the suture element 6. Alternatively to the shown embodiment, the tension sleeve 18, or another tensioning means, which is connected with the suture element 6 and which can be preloaded, can be provided also on a setting device (not shown) connected with the hollow needle 4. The suture element 6 engages in any case on both anchor portions 8, 10, such that they are preloaded in the direction of a deployed position. As can be seen from FIG. 3, the suture element 6 is to this end fastened or looped through respectively at a tension point P of the anchor portions 8, 10.

As can furthermore be seen from FIG. 3, the axle arrangement 12 has an axle element 14 which is provided on the first anchor portion 8 and which engages in a guide recess 16 which is recessed in the second anchor portion 10. The guide recess 16 is in this regard formed as an elongated hole, such that the first anchor portion 8 can be moved rotatably and linearly with respect to the second anchor portion 10.

For setting the anchor 2, a pressing force D is applied to said anchor via an ejector 20 and thereby said anchor is pushed out of the hollow needle 4, while simultaneously the proximally directed tensile force Z acts via the tension points P. Thus results an unfolding moment M by means of which the anchor portions 8, 10 of the anchor 2 at the emergence out of the hollow needle 4 are brought into a deployed position according to FIG. 4.

During the displacement of the anchor portions 8, 10 from the retracted position according to FIGS. 1 and 2 into the deployed position according to FIG. 4, the axle element 14 is here latched in a first latch reception 22, provided on the guide recess 16, as a result of which a pure rotational movement is guaranteed between the anchor portions 8, 10.

As can furthermore be learned from FIG. 4, the suture element 6 in the deployed position engages with the tensile force Z at the two tension points B via two portions which extend in proximal direction obliquely towards each other. As a result, via these two portions force components FL are generated at the two anchor portions 8, 10, which are oriented towards one another in longitudinal direction of the anchor 2. When a specified threshold value is achieved of these longitudinally oriented force components FL, the axle element 14 is released out of the latch connection with the first latch reception 22 and is displaced along the guide recess 16 until it latches with a second latch reception 24, facing away from the first latch reception 22, of the guide recess 16, as shown in FIG. 5.

In this regard, the two anchor portions 8, 10 are displaced towards one another by means of a linear movement until a face-side first abutment surface 26 of the first anchor portion 8 comes into abutment with a face-side second abutment surface 28 of the second anchor portion 10, as shown in FIG. 5. In this position, the anchor portions 8, 10 assume an end position in which they are blocked with regard to a mutual rotational movement by the two abutment surfaces 26, 28 abutting on each other as well as by the axle element 14 latched in the second latch reception 24.

Hereinafter are shown exemplary different embodiments of the anchor 2 or of the axle arrangement 12, by means of which the above-described functionality can be carried out.

As can be seen from FIG. 6, the axle element 14 of the axle arrangement 12 can be designed integrally with the first anchor portion 8. Consistent with this, the guide recess 16 at the second anchor portion 10 can be formed by two longitudinal openings 30, which, spaced parallel to each other, are introduced into respectively one partial portion 32 of the second anchor portion 10. These two partial portions 32 are in this regard spaced from one another such that the axle element 14 of the first anchor portion 8 can be inserted between them, as shown in FIG. 7. By means of a subsequent rotational movement of the first anchor portion 8 around 90° about its own longitudinal axis, the axle element 14 can be brought at both ends into engagement with the two longitudinal openings 30 of the guide recess 16, as shown in FIG. 8.

FIGS. 9 to 11 show an alternative embodiment of the axle arrangement 12, in which the axle element 14 is formed substantially by a pin 34, which is formed separately from a pin reception 36 of the first anchor portion 8. In order to connect the two anchor portions 8, 10, first the pin reception 36 is positioned adjacent to the guide recess 16 of the second anchor portion 10. Here, the pin reception 36, as shown in exemplary manner in FIG. 10, can be formed by two receiving openings, between which the guide recess 16 is inserted. Subsequently, the pin 34 is plugged into the pin reception 36, wherein it simultaneously engages through the guide recess 16 and according to FIG. 11 forms an articulated joint between the two anchor portions 8, 10, by means of which the above described rotational and linear movement between both parts can be undertaken.

A further embodiment of the anchor 2 can be seen from FIGS. 12 and 13. In the figures, the guide recess 16 of the second anchor portion 10 has an elastically extendable introduction opening 38. By means of this introduction opening 38, the axle element, which is either connected as shown integrally with the first anchor portion 8 or can be mounted thereon as a separate pin 34, is introduced into the guide recess 16 and can be accommodated rotatably and linearly movably.

FIGS. 14 to 16 show a further embodiment of the anchor 2, in which the guide recess 16 of the second anchor portion is formed substantially circular. In addition, the guide recess 16 here has a form-fit means 40 in the form of an opening at the edge. To match this, the axle element 14 of the first anchor portion 8 has a cross-section on which a form-fit counter means 42 protrudes in the form of a cam.

In the retracted position shown in FIG. 14, the cam-shaped form-fit counter means 42 is not in engagement with the opening forming the form-fit means 40. Thus, the two anchor portions 8, 10 can be pivoted or rotated relatively to each other into the deployed position shown in FIG. 15 by means of the axle arrangement 12 formed by the axle element 14 and the guide recess 16. In this position, the form-fit means 40 of the second anchor portion 10 is initially disposed adjacent to the form-fit counter means 42 of the first anchor portion 8.

As can be seen from FIG. 16, during the repairing procedure the anchor 2 is laid on the tissue G in this deployed position by applying the tensile force Z to the suture element 6. When the tensile force Z is applied further, there takes place here the linear movement of the second anchor portion 10 with respect to the first anchor portion 8 transversely to the longitudinal extension of both anchor portions 8, 10. As a result, the cam-shaped form-fit counter means 42 of the axle element 14 comes into form-fitting engagement with the form-fit means 40 of the guide recess 16 and therewith into the end position of the anchor 2 according to FIG. 17. In this end position, a rotational movement between the two anchor portions 8, 10 is blocked by the form-fitting connection between the form-fit means 40 and the form-fit counter means 42.

As, for example, can be further seen from FIG. 17, in all above-mentioned embodiments of the anchor 2, receiving openings 44 are introduced into the anchor portions 8, 10 for applying or looping-through the suture element 6. The receiving openings 44 are in this regard positioned such that the suture element 6 provided thereon can be used both for generating the unfolding moment M and for the permanent fastening of the anchor 2 on the tissue G.

Alternatively to this, it is however also possible to provide a two-part suture element 6 having a lifting suture element 46 and a holding suture element 48, as shown in FIGS. 18 to 20. Hereby, the lifting suture element 46 can be connected or redirected at suitable tension points P with the two anchor portions 8, 10, such that when setting the anchor 2 a sufficiently large unfolding moment can be generated in order to bring the anchor portions securely into the deployed position on the tissue G according to FIG. 19. When the tensile force Z is applied further, by means of the lifting suture element 46 and/or the holding suture element 48 the linear movement into the end position according to FIG. 20 can be undertaken.

In this regard, the material of the lifting suture element 46 can be selected such that it can be relatively rapidly reabsorbed after the anchor 2 has been successfully mounted on the tissue G. In contrast thereto, the holding suture element 48 can be mounted on the anchor 2 and formed from a material such that the tensile force required for the repair of the tissue G can be applied to it over a longer period of time, and therewith holds the anchor 2 securely in the end position.

It is noted that all above described elements and characteristics of the different embodiments of the subject matter according to the invention can be exchanged or combined among each other, inasmuch as an exchange or a combination thereof is not excluded for technical reasons.

1. An anchor for the surgical repair of tissue,
said anchor being for placement on the tissue to be repaired and being movable along a hollow needle and connectable to a suture element, and
said anchor having at least two anchor portions displaceable relative to each other, and said at least two anchor portions being connected to each other by an axle arrangement, via which the anchor portions are pivotable relative to each other between a retracted position and a deployed position,
wherein the axle arrangement has an axle element provided on the first anchor portion and a guide recess provided on the second anchor portion by which the first anchor portion is held rotatably and linearly displaceably relative to the second anchor portion, and
wherein the anchor portions are displaceable from the retracted position to the deployed position by a rotational movement and are displaceable from said deployed position to a rotationally blocked end position by a linear movement.

2. The anchor according to claim 1, wherein the axle element is designed integrally with the first anchor portion.

3. The anchor according to claim 1, wherein the axle element is formed by a pin which can be connected with the first anchor portion.

4. The anchor according to claim 1, wherein the guide recess has an elastically extendable introduction opening for introducing the axle element.

5. The anchor according to claim 1, wherein the guide recess is formed by two parallel disposed, spaced longitudinal openings between which the axle element can be inserted and by rotating about 90° can be brought at both ends into guidedly displaceable engagement.

6. The anchor according to claim 1, wherein the axle element and the guide recess are provided at one end of the respective anchor portion.

7. The anchor according to claim 1, wherein the guide recess has an elongated hole along which the axle element can be moved into the end position.

8. The anchor according to claim 7, wherein the axle element can be latched in the retracted position at the elongated hole.

9. The anchor according to claim 7, wherein the axle element can be latched in the end position at the elongated hole.

10. The anchor according to claim 1, wherein the guide recess has a form-fit structure, which in the end position interacts with a form-fit counter structure of the axle element.

11. The anchor according to claim 1, wherein the first anchor portion has at least one first abutment surface, which in the end position can be laid on a second abutment surface of the second anchor portion.

12. The anchor according to claim 1, wherein both anchor portions have respectively a receiving opening at which the suture element can respectively be redirected or fixed.

13. The anchor according to claim 1, wherein the anchor portions in the retracted position have a composite cross-section which extends completely within a virtual enveloping circle with a diameter of less than 2 mm.

14. The anchor according to claim 1, wherein the anchor portions in the end position have a total length of less than 10 mm.

15. An anchor setting arrangement comprising:
the anchor, accommodated in a needle, according to claim 1; and
a suture element,
wherein a tensile force can be applied to the at least two anchor portions by means of the suture element.

16. The anchor setting arrangement according to claim 15, wherein the tensile force can be generated by a displaceable tension sleeve.

17. The anchor setting arrangement according to claim 15, wherein, in addition to the tensile force which can be applied by means of the suture element to both anchor portions a pressing force directed in the opposite direction offset to the tensile force can be applied to said anchor portions in order to generate an unfolding moment.

18. The anchor setting arrangement according to claim 17, wherein the pressing force can be generated by a displaceable ejector in the needle.

19. The anchor setting arrangement according to claim 15, wherein the suture element engages on the two anchor portions with tensile forces directed obliquely towards one another.

20. The anchor setting arrangement according to claim 15, wherein a lifting suture element for displacing the anchor portions out of the retracted position into the deployed position and/or the end position, as well as a holding suture element for fixing the anchor with respect to the tissue to be repaired, are provided.
